SEQUENCE LISTING (2 sequences)

SEQ ID NO: 1 - protein, 7 aa, unidentified
GNNQQNY

SEQ ID NO: 2 - skipped

(19) United States
(12) Patent Application Publication
(10) Pub. No.: US 2025/0298028 A1
(43) Pub. Date: Sep. 25, 2025

(54) METHOD FOR SCREENING DRUGS FOR TREATING MULTIPLE-VARIANT CORONAVIRUS DISEASE

(71) Applicants: KOREA UNIVERSITY RESEARCH AND BUSINESS FOUNDATION, Seoul (KR); KOREA UNIVERSITY RESEARCH AND BUSINESS FOUNDATION, SEJONG CAMPUS, Sejong (KR)

(72) Inventors: Dae Sung YOON, Seoul (KR); Hyo Gi JUNG, Seoul (KR); Dongtak LEE, Seoul (KR); Dongsung PARK, Seoul (KR); Jae Won JANG, Seoul (KR); Yong Hwan KIM, Seoul (KR); Junho BANG, Seoul (KR); Gyudo LEE, Seoul (KR); Da Yeon CHEONG, Sejong (KR); Hyun-ji KIM, Seoul (KR); Jaeheung KIM, Seoul (KR); Young Jun SEO, Uijeongbu-si (KR); Yeon Ho KIM, Anseong-si (KR); Jihye HONG, Seoul (KR); Sang Won LEE, Seoul (KR)

(73) Assignees: KOREA UNIVERSITY RESEARCH AND BUSINESS FOUNDATION, Seoul (KR); KOREA UNIVERSITY RESEARCH AND BUSINESS FOUNDATION, SEJONG CAMPUS, Sejong (KR)

(21) Appl. No.: 18/860,404

(22) PCT Filed: Nov. 23, 2022
(86) PCT No.: PCT/KR2022/018583

§ 371 Date: Oct. 25, 2024

(30) Foreign Application Priority Data
Apr. 28, 2022 (KR) 10-2022-0053001

(51) Int. Cl. G01N 33/68; G01N 21/78

(52) U.S. Cl. CPC G01N 33/68; G01N 21/78; G01N 2333/4709; G01N 2500/04

(57) ABSTRACT

The present disclosure relates to a method for screening drugs for treating multiple-variant coronavirus disease. The present disclosure provides a method for screening coronavirus therapeutic agents in a short period of time by using a low concentration of M^{pro}.

### TECHNICAL FIELD

The present disclosure relates to a method for screening drugs for treating multiple-variant coronavirus disease.

### BACKGROUND ART

Recently, much research has been conducted on vaccines and therapeutic agents to overcome the COVID-19 pandemic, but a spiked protein of SARS-COV-2 continues to mutate. In particular, questions are being raised about the effectiveness of vaccines by research reports showing that antibody levels decrease after vaccination, and clinical trials for therapeutic agents are steadily increasing, and thus research and development for the development of COVID-19 therapeutic agents is urgently needed.

There are greatly three mechanisms for drugs to treat COVID-19, and the first is drugs that target the spiked protein of SARS-COV-2. These drugs bind to the spiked protein to prevent the virus from binding to an ACE-2 receptor on the surface of a host cell, thereby blocking the introduction of the virus into the host cell. However, there is a limitation that the efficacy of neutralizing antibodies is reduced due to frequent mutations in the spiked protein. In particular, the highly infectious Delta variant has 16 mutations within the spiked protein, and the Omicron variant has 32 mutations, making it more difficult for neutralizing antibodies to act.

The second is drugs that target viral RNA. These drugs inhibit viral replication by interfering with a SARS-COV-2 RNA replication process to cause lethal RNA mutations. Remdesivir and Molnupiravir are representative examples, but in addition to controversy over the efficacy of the drugs, it has been reported that Remdesivir has side effects such as lowering liver and kidney functions, and it has been reported that Molnupiravir has a fatal side effect of causing mutations even in mammalian RNA, which may cause birth defects or congenital genetic diseases due to genome damage.

The third is drugs that target a protease derived from SARS-COV-2. As a method for inhibiting the proliferation of the virus by inhibiting the activity of M^{pro}, the main protease of SARS-COV-2, specifically, it is a viral protease that initiates replication by cleaving a pp1a/ab protein of the virus that has entered the cell. Meanwhile, a viral replication mechanism by M^{pro} is shared with various types of coronaviruses (SARS-COV, MERS-COV, HCoV-HKU1, etc.). When comparing an M^{pro} active site sequence of SARS-COV-1, which was prevalent in 2003, with an M^{pro} active site sequence of SARS-COV-2 in 2021, no mutations were observed. Accordingly, drugs targeting the M^{pro} active site are expected to have low concerns about side effects on the human body and have a very high possibility of being used as coronavirus therapeutic agents that may occur in the future.

Accordingly, the present disclosure provides a method for screening an M^{pro} inhibitor that inhibits the activity of M^{pro} of a coronavirus. In particular, conventional methods for screening an M^{pro} inhibitor not only used a high concentration of M^{pro} despite the high unit price of M^{pro}, but also required two or more fluorescent substances, and took several days for screening. To overcome these limitations, a method is developed and provided to screen coronavirus therapeutic agents in a short period of time using a low concentration of M^{pro}.

### DISCLOSURE

### Technical Problem

An object of the present disclosure is to provide a method for screening drugs targeting an M^{pro} active site that is not modified even with mutations of coronavirus.

Another object of the present disclosure is to provide a composition for screening coronavirus therapeutic agents.

### Technical Solution

An aspect of the present disclosure provides a method for preparing a composition for screening coronavirus therapeutic agents comprising: (a) preparing an engineered amyloid peptide comprising a sequence represented by SEQ ID NO: 1 and SEQ ID NO: 2; (b) preparing gold nanoparticles by mixing hydrogen tetrachloroaurate (HAuCl₄) and sodium citrate; and (c) preparing an engineered amyloid nanocomposite by mixing the amyloid peptide of step (a) and the gold nanoparticles of step (b).

In the present disclosure, the engineered amyloid peptide of step (a) above may be a β-sheet, but is not limited thereto.

In the present disclosure, step (b) may be heating a mixture of hydrogen tetrachloroaurate and ultrapure water, and then adding sodium citrate and heating the mixture, but is not limited thereto.

In the present disclosure, the engineered amyloid nanocomposite of step (c) may be prepared by mixing gold nanoparticles and an engineered amyloid peptide in a volume ratio of 1:0.5 to 10, but is not limited thereto.

In the present disclosure, the engineered amyloid nanocomposite of step (c) above may be red, but is not limited thereto.

Another aspect of the present disclosure provides a composition for screening coronavirus therapeutic agents prepared by the preparation method.

Yet another aspect of the present disclosure provides a method for screening for coronavirus therapeutic agents, including treating the composition with a coronavirus therapeutic agent candidate drug.

In the present disclosure, the screening method may further include selecting the candidate drug as a coronavirus therapeutic agent if a color changes to blue after the candidate drug treatment, but is not limited thereto.

### Advantageous Effects

According to present disclosure, it is possible to provide a method capable of screening coronavirus therapeutic agents in a short period of time using a low concentration of M^{pro}.

### DESCRIPTION OF DRAWINGS

FIG. 1 is a diagram briefly illustrating amyloid peptide synthesis principle and screening principle of the present disclosure.

FIG. 2 is a diagram illustrating a result of analyzing fibrillation of an amyloid peptide in one embodiment of the present disclosure. FIG. 2A illustrates an atomic force microscopy (AFM) image result of a MCAP monomer, FIG. 2B is a graph showing heights of white dotted lines in FIG. 2A, FIG. 2C is a graph showing an average size of MCAP monomer analyzed through an AFM graph, FIG. 2D is an AFM image result of a fibrillated amyloid peptide, FIG. 2E is a graph showing a height of a white dotted line in FIG. 2D, FIG. 2F is a graph showing an average size of a fibrillated amyloid peptide analyzed through an AFM graph, and FIG. 2G is a transmission electron microscope (TEM) image result of a fibrillated amyloid peptide and an enlarged view thereof.

FIG. 3 is a diagram confirming a β-sheet structure of a fibrillated amyloid peptide in one embodiment of the present disclosure. FIG. 3A shows a result of dynamic light scattering (DLS) analysis, FIG. 3B shows a result of circular dichroism (CD) analysis, FIG. 3C shows a result of Thioflavin T fluorescence analysis, and FIG. 3D shows a result of FT-IR analysis.

FIG. 4 is a diagram confirming that a fibrillated amyloid peptide of the present disclosure is decomposed according to M^{pro} treatment.

FIG. 5 is a diagram analyzing the structural stability of an engineered amyloid nanocomposite (MCAP-AuNP) in one embodiment of the present disclosure. FIG. 5A is a diagram showing absorbance peaks according to a mixing amount of an amyloid peptide solution. FIG. 5B shows a result of time-specific UV-vis spectrum analysis of a nanocomposite prepared with a 50 μL amyloid peptide solution.

FIG. 6 is a diagram analyzing the freeze-thaw performance of an engineered amyloid nanocomposite (MCAP-AuNP) in one embodiment of the present disclosure. FIG. 6A shows a result of UV-vis spectrum analysis before freezing, and FIG. 6B shows a result of UV-vis spectrum analysis after freezing and thawing. FIGS. 6C and 6D show color changes of a gold nanoparticle (bare AuNP, left: before freezing, right: after freezing and thawing) and an engineered amyloid nanocomposite (MCAP-AuNP, left: before freezing, right: after freezing and thawing) before and after freezing and thawing, respectively.

FIG. 7 is a diagram analyzing physical and chemical properties of a gold nanoparticle (bare AuNP) and an engineered amyloid nanocomposite (MCAP-AuNP) in one embodiment of the present disclosure. FIG. 7A shows a result of measuring a hydrodynamic diameter, FIG. 7B shows a result of analyzing a zeta potential, FIG. 7C shows a result of confirming by X-ray photoelectron spectroscopy that amyloid peptides are coated on the surface of gold nanoparticles of the engineered amyloid nanocomposite (MCAP-AuNP), FIG. 7D shows a TEM image of the engineered amyloid nanocomposite (MCAP-AuNP), and FIG. 7E shows the structural analysis of MCAP-AuNP using FT-IR, and a result of confirming that MCAP aggregates coated on the surface of MCAP-AuNP are in a beta structure form.

FIG. 8A is a diagram showing the analysis of a UV-vis spectrum of an engineered amyloid nanocomposite (MCAP-AuNP) according to a concentration of M^{pro} in one embodiment of the present disclosure, FIG. 8B is a diagram showing a result of the same analysis using modified M^{pro}, FIG. 8C is a graph showing an S-shaped dose-response curve quantifying the degree of aggregation of MCAP-AuNP (A₆₅₀/A₅₂₅) depending on modification of M^{pro}, and FIG. 8D is a diagram showing a result of a TEM image of MCAP-AuNP depending on modification of M^{pro} treated with MCAP-AuNP. FIG. 8E is a diagram showing selectivity for MCAP-AuNP, and a diagram confirming that MCAP-AuNP selectively reacts to M^{pro}. FIG. 8F is a diagram showing reactivity according to a concentration of M^{pro}, and a diagram confirming that the MCAP-AuNP of the present disclosure has significantly better reactivity than a commercialized fluorescence-based M^{pro} activity measurement kit.

FIG. 9 is a diagram showing the analysis of a UV-vis spectrum of an engineered amyloid nanocomposite (MCAP-AuNP) by screening previously known M^{pro} inhibitors in one embodiment of the present disclosure.

FIG. 10A is a diagram showing an image obtained by observing a reaction of an engineered amyloid nanocomposite (MCAP-AuNP) according to M^{pro} treatment using TEM in one embodiment of the present disclosure. FIG. 10B shows a reaction result according to leupeptin treatment, FIG. 10C shows a reaction result according to lopinavir treatment, and FIG. 10D shows a reaction result according to hesperetin treatment.

FIG. 11 is a diagram showing the difference between an engineered amyloid peptide (MCAP) and an engineered peptide MCRP that does not include an amyloid sequence, in one embodiment of the present disclosure.

FIG. 12 is a diagram showing the analysis of ThT signals of an engineered amyloid peptide (MCAP) and MCRP in one embodiment of the present disclosure.

FIG. 13 is a diagram analyzing enzyme activity of M^{pro} according to treatment with M^{pro} inhibitors in one embodiment of the present disclosure.

### BEST MODE OF THE INVENTION

The present disclosure relates to an engineered amyloid nanocomposite (MCAP-AuNP) in which an amyloid peptide containing a M^{pro} cleavage sequence (L-Q-S) is coated on a gold nanoparticle, and to a method for screening an M^{pro} inhibitor that inhibits M^{pro}, a coronavirus-derived protease. The M^{pro} (main protease) is a coronavirus-derived protease, and the present disclosure may inhibit the proliferation of the virus by inhibiting the virus-derived protease. In the present disclosure, the coronavirus refers to viruses belonging to the coronavirus family. Coronaviruses are known to cause respiratory or gastrointestinal infections, depending on the characteristics and a host of the virus.

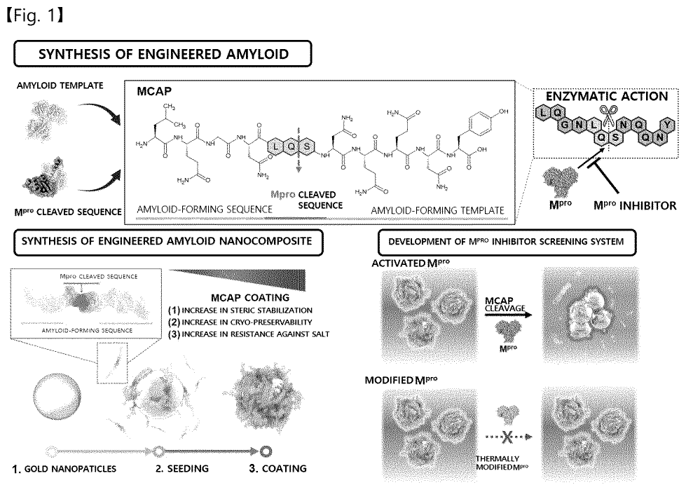

When the engineered amyloid nanocomposite of the present disclosure is treated with M^{pro} and a specific drug, if the specific drug includes an M^{pro} inhibitory effect, no change in the color of a solution is observed, and if the specific drug does not include the M^{pro} inhibitory effect, a change in the color of the solution is observed. In the solution, a color change occurs due to structural stability collapse, such as aggregation of gold nanoparticles, and the M^{pro} cleavage sequence (L-Q-S) is cleaved by M^{pro} to decompose the amyloid peptide and the aggregation of gold nanoparticles is induced to cause a color change in the solution. On the other hand, M^{pro} having activity reduced by the M^{pro} inhibitor, does not decompose the amyloid peptide and does not induce the aggregation of gold nanoparticles, so that no color change in the solution is observed (see FIG. 1).

Meanwhile, a viral replication mechanism by M^{pro} is shared with various types of coronaviruses (SARS-COV, MERS-COV, HCoV-HKU1, etc.). When comparing an M^{pro} active site sequence of SARS-COV-1, which was prevalent in 2003, with an M^{pro} active site sequence of SARS-COV-2 in 2021, no mutations were observed. Accordingly, drugs targeting the M^{pro} active site are expected to have low concerns about side effects on the human body and have a very high possibility of being used as coronavirus therapeutic agents that may occur in the future.

### Modes of the Invention

Hereinafter, the present disclosure will be described in more detail through Examples and Experimental Examples. However, the following Examples and Experimental Examples are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

### Preparation Example 1

A main protease (M^{pro}) of lyophilized SARS-COV-2 was purchased from Biosynth Carbosynth (UK). Ebselen, hesperetin, leupeptin, lopinavir, hesperidin, chloroauric acid trihydrate (HAuCl₄·3H₂O) and trisodium citrate were purchased from Sigma-Aldrich (USA). Distilled water (DW) and phosphate buffered saline (PBS) were purchased from Gibco (USA).

### <Example 1> Preparation of Amyloid Peptide

A main protease (M^{pro}) of SARS-COV-2 cleaved L-Q-S, L-Q-A, and L-Q-G sequences. In this experiment, an amyloid sequence (G-N-N-Q-Q-N-Y) derived from a prion protein and a M^{pro} cleavage sequence (L-Q-S) were fused to form an engineered amyloid peptide (M^{pro} cleavage site embedded amyloid peptide, MCAP, L-Q-G-N-L-Q-S-N-Q-Q-N-Y, Peptron, Korea) from the C-terminus using a Solid Phase Peptide Synthesis method (FIG. 1).

TABLE 1

| | |
|---|---|
| SEQ ID NO: 1 | GNNQQNY |
| SEQ ID NO: 2 | LQS |

### <Example 2> Fibrillation of Engineered Amyloid Peptide (MCAP)

### 2-1. Fibrillation of MCAP

An engineered amyloid peptide (MCAP) containing a prion protein-derived amyloid sequence (G-N-N-Q-Q-N-Y) and an M^{pro} cleavage sequence (L-Q-S) was dissolved in distilled water to prepare a 1 mg/mL MCAP solution. 50 μL of the MCAP solution and 150 μL of distilled water (pH 2) were mixed and sufficiently reacted in a 37° C. shaking incubator (Eppendorf, Germany) at 1,000 rpm for 5 days to be fibrillated. Fibrillated amyloid peptides were identified as follows.

### 2-2. AFM (Atomic Force Microscope) and TEM (Transmission Electron Microscope) Analysis

A silicon wafer was washed with a piranha solution (H₂SO₄:H₂O₂=1:1). 50 μL of a fibrillated amyloid peptide solution was deposited on the silicon wafer for 20 minutes at room temperature, washed with distilled water, and then dried in a fume hood for 12 hours.

AFM analysis was performed with an NX10 (Park systems, South Korea) using a silicon tip with a radius of less than 10 nm (NCHR, Park Systems, South Korea). AFM measurement was performed in an NCM mode at a scan rate of 0.4 Hz and an image size of 5 μm×5 μm. Image flattening and topological analysis were performed using Smart Scan (Park systems, South Korea) software. Images obtained through AFM analysis were used to measure the persistence length of the fibrillated amyloid peptide using Easyworm software.

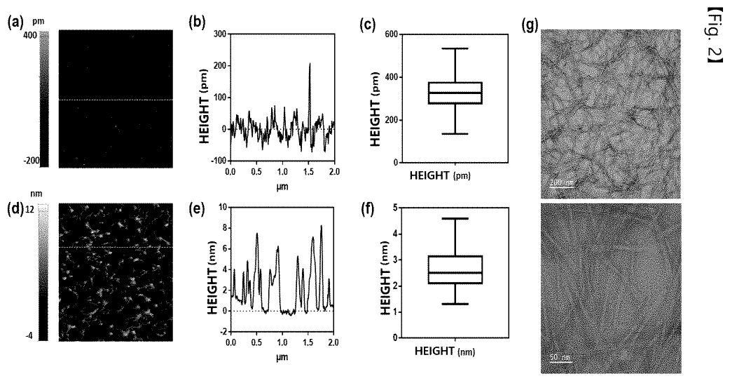

FIG. 2A shows an AFM image analyzing the persistence length of the MCAP monomer. FIG. 2B is a diagram showing the height of a white dotted line in FIG. 2A. FIG. 2C shows an approximate average size of the MCAP monomer analyzed through AFM images, which is 330.19±73.9 pm. FIG. 2D shows an AFM image analyzing the persistence length of a fibrillated amyloid peptide. FIG. 2E is a diagram showing the height of a white dotted line in FIG. 2D. FIG. 2F shows an approximate average size of the fibrillated amyloid peptide analyzed through AFM images, which is 2.64±0.74 pm.

TEM analysis was performed after reacting 0.25 mg/mL of a MCAP solution at 37° C. and 1,000 rpm for 96 hours, centrifuging at 12,000 rpm for 1 hour, and removing the supernatant. FIG. 2G is a TEM analysis image and its enlarged image. Through this, it was confirmed that the fibrillation of MCAP had occurred.

### <Example 3> Confirmation of β-Sheet Structure of Fibrillated Amyloid Peptide

### 3-1. Dynamic Light Scattering (DLS) Analysis

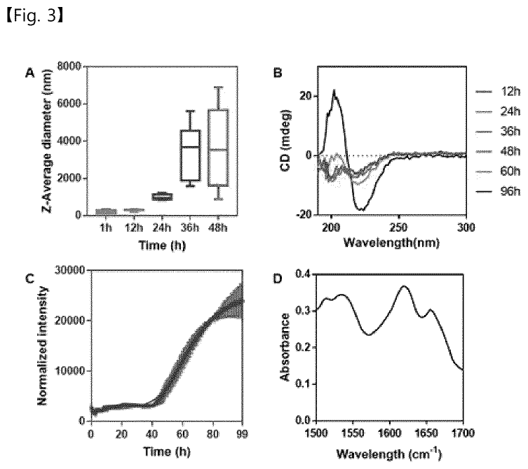

A hydrodynamic diameter was measured using dynamic light scattering (DLS) while reacting 0.25 mg/mL of an MCAP solution at 37° C. and 1,000 rpm for 3 days. 1 mL of the reacted MCAP solution was placed in a disposable cuvette (10 mm light path, standard type, Kartell, Italy) and measured 40 times in total, 10 times per cycle for 4 cycles, using a Zetasizer Nano S90 (Malvern Panalytical, United Kingdom). As shown in FIG. 3A, fibrillation progressed from a monomeric form of MCAP depending on a reaction time, and the diameter and standard deviation increased (1 h: 0.20±0.073 μm, 12 h: 0.30±0.03 μm, 24 h: 0.98±0.13 μm, 36 h: 3.40±1.37 μm, 48 h: 3.62±2.02 μm).

### 3-2. Circular Dichroism (CD) Analysis

CD may identify the secondary structure of a material, and a random coil form shows a negative y-axis value at a wavelength≤210 nm. 0.25 mg/ml of the MCAP solutions reacted at 37° C. and 1,000 rpm for 12, 24, 36, 48, 60, and 96 hours, respectively, and then was injected into a quartz glass cuvette (1 mm path length, 10 mm inside wide, Aireka Cells, USA) and measured. Circular dichroism spectra were measured using J-815 (Jasco, Japan) with a spectral detection range of 190 to 300 nm and a scanning speed of 10 nm/min. The spectra had a resolution of 8 nm and were processed with CDTool software. As shown in FIG. 3B, the fibrillated amyloid peptide showed a peak with a negative value at a wavelength of 210 to 240 nm and a positive value at a wavelength≤210 nm. That is, the signal due to the β-sheet structure increased according to a reaction time, and it was meant that MCAP was gradually fibrillated into the β-sheet structure.

### 3-3. Thioflavin T Fluorescence Analysis

Thioflavin T (ThT) was a fluorescent substance that detected a β-sheet structure. 0.25 mg/mL of the MCAP solution reacted with 20 μM of a ThT solution in a pH 2 solution at 37° C. and 1,000 rpm for 3 days, and a ThT fluorescence signal was measured. The ThT fluorescence signal was measured at 30-minute intervals for 99 hours. The experimental result was shown in FIG. 3C. The ThT fluorescence signal value increased with reaction time, which indicated that a fibrillated amyloid peptide in a β-sheet form was formed.

### 3-4. Fourier Transform Infrared (FT-IR) Analysis

When measuring the β-sheet structure, FT-IR confirmed a peak around 1,620/cm. A fibrillated amyloid peptide solution reacted for 48 hours was centrifuged at 12,000 rpm for 1 hour and the supernatant was removed. The fibrillated amyloid peptide solution was deposited on a silicon wafer and then dried in a fume hood for 12 hours. FT-IR spectra were measured using a Cary 630 FTIR Spectrometer (scanning range 1,600 to 1,700/cm, Agilent Technologies, USA). The spectra had a resolution of 4 nm and were processed with Agilent MicroLab software. As shown in FIG. 3D, a peak was measured around 1,620/cm, and thus it was confirmed that the fibrillated amyloid peptide had a β-sheet structure.

### <Example 4> Confirmation of Decomposition of Fibrillated Amyloid Peptide by M^{pro}

### 4-1. Atomic Force Microscope (AFM) Analysis

The decomposition of fibrils was confirmed by reacting M^{pro} with an engineered amyloid peptide (MCAP) in Example 2. 0.25 mg/ml of the MCAP solution was incubated at pH 2 and 37° C. for 5 days to prepare a fibrillated amyloid peptide. The prepared fibrillated amyloid peptide reacted with 0.01 mg/ml of M^{pro} for 12 hours.

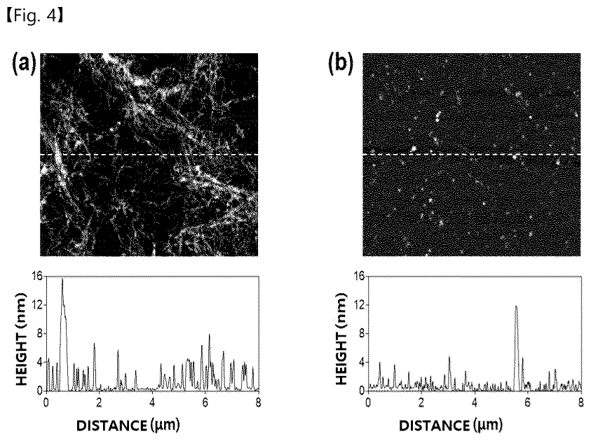

As shown in FIG. 4A, it was confirmed that the fibrillated amyloid peptide was prepared. As shown in FIG. 4B, it was confirmed that when 0.01 mg/ml of M^{pro} reacted with the fibrillated amyloid peptide for 12 hours, the fibrils were decomposed and broken into small pieces to be changed into the form of small lumps. The heights of the white dotted lines in the image were graphed and shown below, respectively.

### <Example 5> Synthesis of Engineered Amyloid Nanocomposite (MCAP-AuNP)

### 5-1. Preparation of Gold Nanoparticles (AuNP)

Gold nanoparticles were synthesized using citrate reduction. 2.5 mL of a 38.8 mM hydrogen tetrachloroaurate (HAuCl₄) solution and 45 mL Millipore water were mixed in a round beaker and heated while stirring at 1,200 rpm. After boiling the mixture, 1 mL of 80 mM sodium citrate solution was added and heated for 1 hour while stirring at 1,200 rpm to prepare gold nanoparticles stabilized with citric acid. The gold nanoparticles were prepared with a size of 20 nm. The final gold nanoparticle solution was cooled to room temperature and stored at 4° C.

### 5-2. Optimization of Engineered Amyloid Nanocomposite (MCAP-AuNP)

0.1 mg/mL of a MCAP solution was prepared by adding a mixture of distilled water and PBS in a volume ratio of 10:1 to MCAP. A 20 nm gold nanoparticle solution was centrifuged at 13,000 rpm for 20 minutes, and the supernatant was removed. 40 μL of the gold nanoparticle solution was mixed with 25, 50, 100, 200, 300, and 400 μL (0.09 mg/mL) of Example 2, respectively, and reacted at 37° C. and 1,000 rpm to synthesize an engineered amyloid nanocomposite. A salt resistance test was performed for 24 hours by adding 600 μL of 1×PBS, and a UV-vis spectrum was measured. The UV-vis spectrum was measured using a spectrophotometer (scan range 400 to 800 nm, scan rate 600 nm/min, Perkin Elmer, USA). The UV-vis spectrum of the dispersed gold nanoparticles of 20 nm or less had a peak detected around 525 nm (A₅₂₅). When the fibrillated amyloid peptide did not coat the surface of the gold nanoparticle well, the gold nanoparticles were aggregated by PBS to cause a color change in the solution, and the peak shifted to near 650 nm (A₆₅₀). Therefore, the degree of aggregation of gold nanoparticles may be quantified by relative absorbance (A₆₅₀/A₅₂₅).

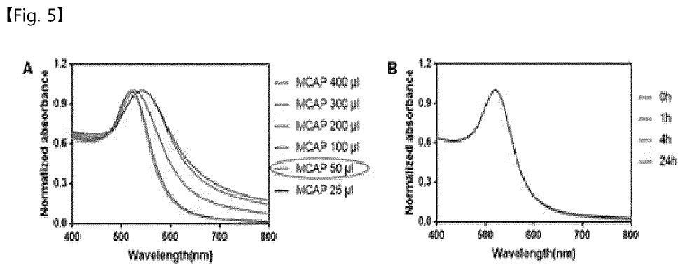

As shown in FIG. 5A, when 25 to 100 μL of the MCAP solution was used, the peak was observed around 525 nm (A₅₂₅), and thus confirmed as the most stable structure.

Meanwhile, in order to further confirm the structural stability of the nanocomposite prepared with 50 μL of the MCAP solution, distilled water was added to the nanocomposite to make a total volume 0.8 mL, and the UV-vis spectrum was measured over time. As a result, as shown in FIG. 5B, it was confirmed that the structural stability was maintained for 24 hours.

### <Example 6> Characterization Analysis of Engineered Amyloid Nanocomposite (MCAP-AuNP)

### 6-1. Freeze-Thaw Performance Analysis

In this experiment, the freeze-thaw performance of the engineered amyloid nanocomposite (MCAP-AuNP) prepared with the gold nanoparticle solution (bare AuNP) prepared in Example 5 and 50 μL of the MCAP solution was analyzed.

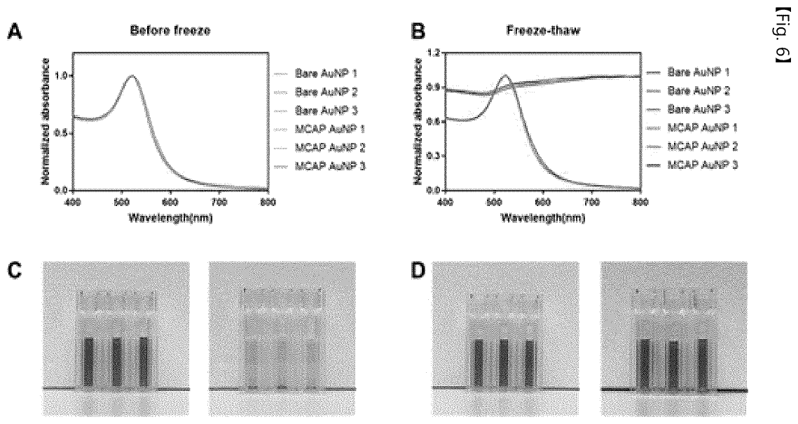

Each 1 mL of bare AuNP and MCAP-AuNP were frozen at −80° C. for 2 hours, and then thawed at room temperature for 2 hours. A hydrodynamic diameter before and after freezing and thawing was measured using a Zetasizer. In addition, the UV-vis spectra of each solution were measured before and after freezing and thawing. The experimental results were shown in FIG. 6.

FIG. 6A shows results of UV-vis spectrum analysis before freezing, and FIG. 6B shows results of UV-vis spectrum analysis after freezing and thawing. As shown in the peak changes before and after freezing and FIG. 6C, aggregation of gold nanoparticles occurred and color changes were observed after freezing and thawing, whereas the engineered amyloid nanocomposite (MCAP-AuNP) showed a constant peak before and after freezing, and as shown in FIG. 6D, no color changes were observed after freezing and thawing, and thus, it was confirmed that structural stability was excellent.

### 6-2. Analysis of Physical and Chemical Properties

In this experiment, the physical and chemical characteristics of the engineered amyloid nanocomposite (MCAP-AuNP) prepared with the gold nanoparticle solution (bare AuNP) prepared in Example 5 and 50 μL of an MCAP solution were analyzed.

As in Example 3 above, the results of measuring the hydrodynamic diameters of bare AuNP and MCAP-AuNP using dynamic light scattering (DLS) were shown in FIG. 7A. The bare AuNP was 19.01±0.53 nm and the MCAP-AuNP was 21.96±0.67 nm, and it was confirmed that the MCAP-AuNP coated with the engineered amyloid peptide was approximately 3 nm thicker than the gold nanoparticle before coating.

A zeta potential value of MCAP-AuNP was shown in FIG. 7B. 0.4 mg/mL of a MCAP-AuNP solution was placed in a Zetasizer Cuvette, and the accumulation time was measured 15 times for each point. As a result, the zeta potential of MCAP-AuNP was −19±3.1 mV, and thus it was confirmed that MCAP-AuNP had excellent structural stability.

Peaks of gold (Au) and nitrogen (N) elements were confirmed using X-ray photoelectron spectroscopy (XPS). The MCAP-AuNP solution was centrifuged at 6,720×g for 20 minutes, and the supernatant was removed. Pellets of each solution (<40 μL) were deposited on the silicon wafer and dried in a fume hood for 24 hours. XPS was measured using a K-alpha instrument (Thermo VG, UK). The instrument may control a monochromatic X-ray source (Al Kα line: 1486.6 eV) at 4.8×10⁻⁹ mb. The range of 0 to 800 eV was scanned using a pass energy of 40 eV with a 0.1 eV step size. As shown in FIG. 7C, a nitrogen (N) Is peak was measured by the protein, and from this, it was confirmed that the gold nanoparticle surface of the engineered amyloid nanocomposite (MCAP-AuNP) was coated with the MCAP solution, i.e., the amyloid peptide.

Filter paper was cut to an appropriate size and placed on the bottom of a petri dish, and a TEM grid was placed thereon. The MCAP-AuNP solution was dropped onto a TEM grid and dried in the petri dish, and then TEM images were taken. FIG. 7D is a TEM image result of the engineered amyloid nanocomposite (MCAP-AuNP). These results also indicate that the engineered amyloid peptide is coated on the surface of the gold nanoparticle of the engineered amyloid nanocomposite (MCAP-AuNP).

An experiment was conducted to analyze the structure of MCAP aggregates coated on the surface of the gold nanoparticle in MCAP-AuNP. In the MCAP-AuNP solution, the MCAP aggregates, that were not coated on the surface of the gold nanoparticle, but existed in the solution, were removed through centrifugation, and the FT-IR absorbance of MCAP-AuNP was measured. At this time, as in FIG. 7E, peaks may be observed in the wavelength range of 1620 to 1630, which means that the MCAP-AuNP has a beta structure. Therefore, it was confirmed through FT-IR absorbance measurement that the MCAP aggregates coated on the MCAP-AuNP surface had a beta-structure form.

### <Example 7> Kinetic Analysis of M^{pro} by Concentration

M^{pro} was dissolved in PBS for each concentration (0.925 to18.5 nM) for 20 minutes, and each volume was prepared at 0.8 mL. The engineered amyloid nanocomposite (MCAP-AuNP) prepared with the gold nanoparticle solution (bare AuNP) prepared in Example 5 above and 50 μL of the amyloid peptide solution was mixed with a M^{pro} solution for each concentration, so that the total volume became 1 mL. After reacting the MCAP-AuNP and M^{pro} mixture at 37° C. for 1 hour, the UV-vis spectrum was measured using a spectrophotometer.

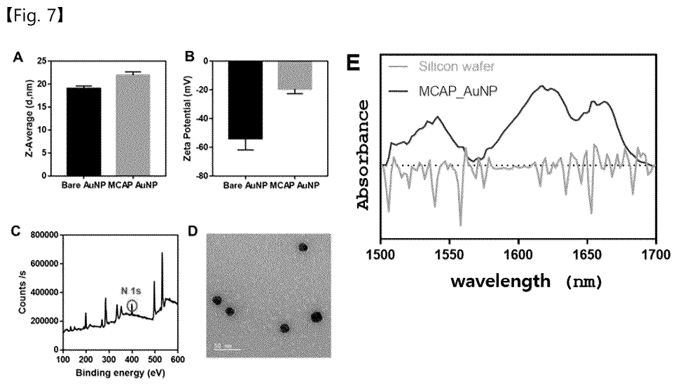

As shown in FIG. 8A, as the M^{pro} concentration increased, the UV peaks shifted to the right. Meanwhile, when M^{pro} was modified at 90° C. for 4 hours and reacted in the same manner as above, it was confirmed that there was no change in the UV spectrum, as shown in FIG. 8B. A S-shaped dose-response curve was shown in FIG. 8C by quantifying (A₆₅₀/A₅₂₅) the degree of aggregation of MCAP-AuNPs depending on the presence or absence of M^{pro} modification. FIG. 8D is a TEM image result of MCAP-AuNP depending on the presence or absence of M^{pro} modification.

In addition, a selectivity experiment was performed on MCAP-AuNP. When MCAP-AuNP reacted with M^{pro}, aggregation occurred and a relative absorbance change occurred, but MCAP-AuNP did not react with modified M^{pro}, so that aggregation did not occur and a relative absorbance change did not occur. A selectivity experiment was conducted to determine whether the MCAP-AuNP of the present disclosure reacted with M^{pro}, and the biomaterials used were human serum albumin, glucose, immunoglobulin G, and bovine serum albumin, which were abundantly present in the body. As a result of reacting the MCAP-AuNP of the present disclosure with the biomaterial for 24 hours and measuring the absorbance, as shown in FIG. 8E, it can be confirmed that an absorbance difference is relatively much lower than that of M^{pro}, which indicates that MCAP-AuNP selectively reacts with M^{pro}.

In addition, the reactivity according to a concentration of M^{pro} was compared using a commercialized fluorescence-based M^{pro} activity measurement kit and the MCAP-AuNP of the present disclosure. The fluorescence-based M^{pro} activity measurement kit was purchased from Sigma Aldrich (MCA-AVLQSGFR-Lys (Dnp)-Lys-NH2 trifluoroacetate). In FIG. 8F, a left y-axis of the graph represents a relative absorbance change according to a M^{pro} concentration measured with MCAP-AuNP, and a right y-axis represents a fluorescence signal intensity change measured with the commercial kit. As a result, it can be confirmed that MCAP-AuNP reacts with a much lower concentration of M^{pro} than that of the commercial kit, and a signal deviation is also much smaller. When this was quantified, the half maximal effective concentration (EC50) for M^{pro} was 4.4 nM for MCAP-AuNP and 211 nM for the commercial kit, and thus it was confirmed that MCAP-AuNP reacted with a 50-fold lower concentration of M^{pro} than that of the commercial kit. Through this, it was confirmed that the MCAP-AuNP of the present disclosure had superior reactivity for M^{pro} to the commercialized kit.

### <Example 8> M^{pro} Inhibitor Screening

M^{pro} inhibitors (ebselen, hesperidin, hesperetin, lopinavir, leupeptin) were dissolved in various concentrations in PBS (1.5% DSMO), and added with 50 ng of M^{pro} (1.4 nM) to prepare a total solution volume of 800 μL. The mixture was reacted at room temperature for 20 minutes, and then filtered through a 200 μm-size PVDF syringe filter (Biopil, China) to remove unreacted substances. 600 μL of the final solution was added to 200 μL of an engineered amyloid nanocomposite (MCAP-AuNP) solution and reacted at 37° C. for 1 hour. After the reaction, the UV-vis spectrum was measured using a spectrophotometer. The degree of particle aggregation was analyzed by relative absorbance (A₆₅₀/A₅₂₅).

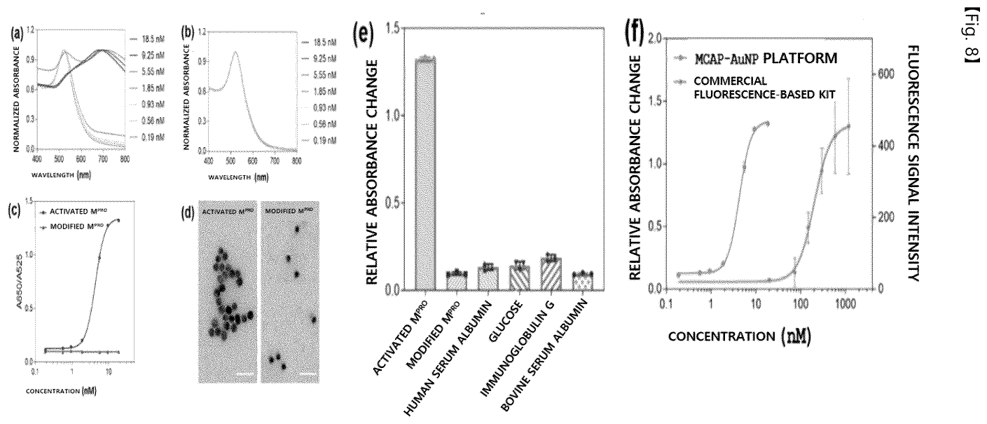

As shown in FIG. 9, the IC₅₀ values of each drug were confirmed as ebselen 0.39 μM, hesperetin 43.14 μM, leupeptin 28.57 μM, lopinavir 10.7 μM, and hesperidin 369.4 μM.

To verify the action of an M^{pro} inhibitor, 12 μg of M^{pro} (1.4 nM) was added to M^{pro} inhibitors (leupeptin, lopinavir, hesperetin) dissolved at various concentrations in PBS (10% DSMO), and the total volume of the solution was prepared to 500 μL. The mixture was reacted at room temperature for 20 minutes, and then filtered through a 200 μm-size PVDF syringe filter (Biopil, China) to remove unreacted substances. Thereafter, 70 μL of the mixture was added to the MCAP solutions of 1.88, 3.7, 5.66, 7.55, 9.43, and 11.32 μM to make a final volume of 220 μL. The relative absorbance (A₆₅₀/A₅₂₅) by the M^{pro} inhibitor was analyzed according to the concentration of the engineered amyloid nanocomposite (MCAP-AuNP) solution using a microplate reader (Molecular Device, USA). As shown in FIG. 13, enzyme activity according to inhibitor treatment was analyzed using a Michaelis-Menten equation.

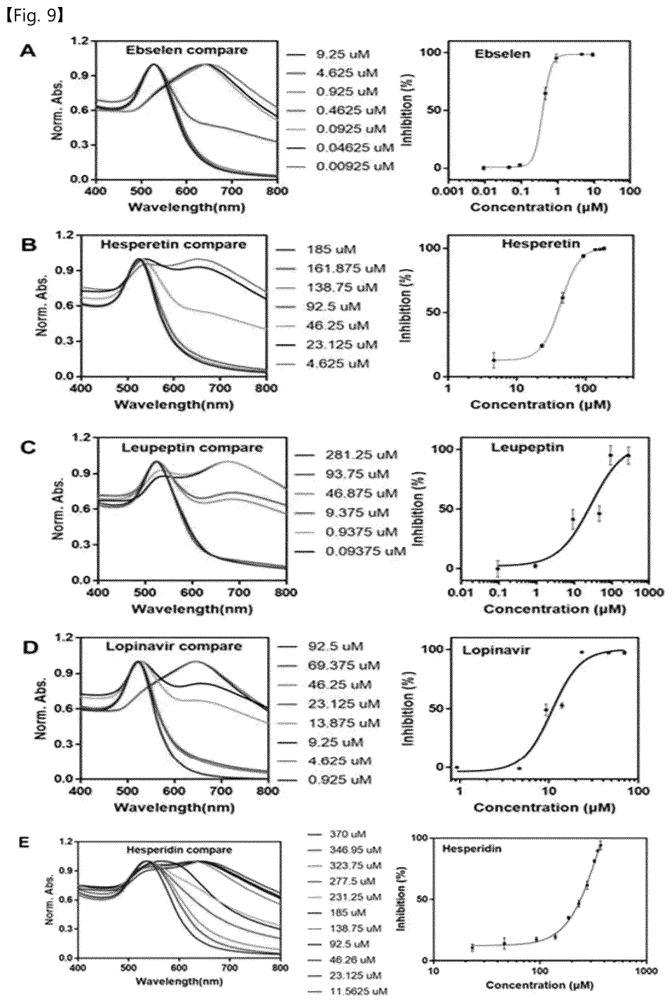

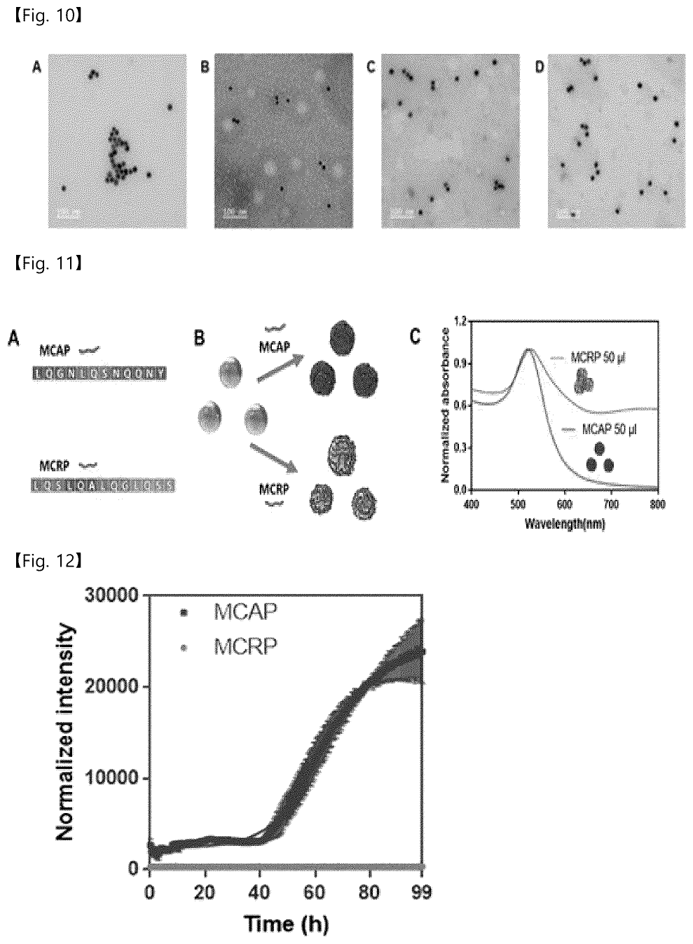

FIG. 10 shows a TEM image of the reaction of an engineered amyloid nanocomposite (MCAP-AuNP) according to M^{pro} treatment. FIG. 10A shows an engineered amyloid nanocomposite (MCAP-AuNP) reacted with M^{pro}, FIG. 10B shows a reaction result according to leupeptin treatment, FIG. 10C shows a reaction result according to lopinavir treatment, and FIG. 10D shows a reaction result according to hesperetin treatment, which are images observed using TEM. As shown in FIG. 10, it was confirmed that the activity of M^{pro} was inhibited by the M^{pro} inhibitor treatment, and thus did not affect the engineered amyloid nanocomposite (MCAP-AuNP).

### <Example 9> Confirmation of Characteristics of Engineered Amyloid Peptides According to Sequence Cleaved by M^{pro}

In this experiment, sequences L-Q-S, L-Q-A, and L-Q-G, which were cleaved by M^{pro}, were connected to prepare a peptide with a sequence L-Q-S-L-Q-A-L-Q-G-L-Q-S-S(M^{pro} cleavage-site-embedded repeated peptide, MCRP). A difference from the engineered amyloid peptide of the present disclosure (M^{pro} cleavage site embedded amyloid peptide, MCAP, L-Q-G-N-L-Q-S-N-Q-Q-N-Y) was shown in FIG. 11A and FIG. 11B.

As the same as in Example 5 above, a nanocomposite was prepared by mixing 50 μL of a MCRP solution and 40 μL of a gold nanoparticle solution, and added with 600 μL of 1×PBS to perform a salt resistance test for 24 hours. The results of measuring the UV-vis spectrum were shown in FIG. 11C. As a result of the experiment, it was confirmed that the structural stability of the nanocomposite was unstable compared to the MCAP of the present disclosure, and in particular, it was confirmed that the absorbance shifted to the right as the gold nanoparticles were aggregated because MCRP did not coat the surface of the gold nanoparticles. That is, MCRP did not have the amyloid properties of a β-sheet and thus could not coat the surface of gold nanoparticles.

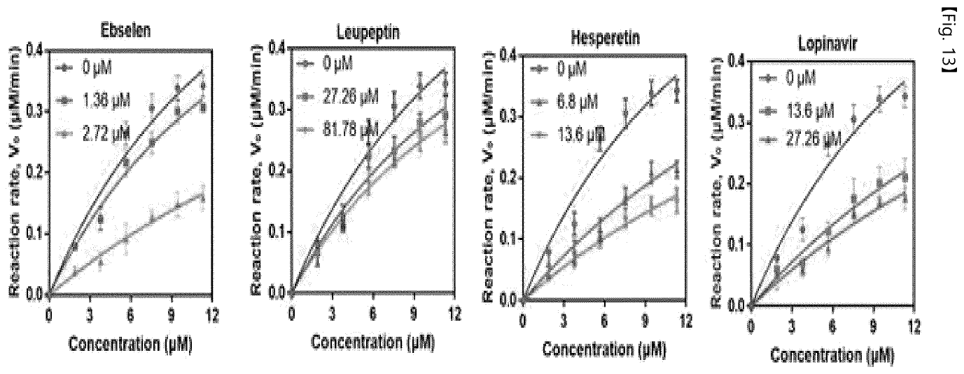

In addition, as a result of performing ThT analysis as in Example 3 above, as shown in FIG. 12, no ThT signal was confirmed compared to the MCAP of the present disclosure, and it was confirmed that this was because the fibrillated amyloid peptide of the β-sheet was not formed.

1. A method for preparing a composition for screening coronavirus therapeutic agents comprising:
(a) preparing an engineered amyloid peptide comprising a sequence represented by SEQ ID NO: 1 and SEQ ID NO: 2;
(b) preparing gold nanoparticles by mixing hydrogen tetrachloroaurate (HAuCl₄) and sodium citrate; and
(c) preparing an engineered amyloid nanocomposite by mixing the amyloid peptide of step (a) and the gold nanoparticles of step (b).

2. The method for preparing the composition for screening coronavirus therapeutic agents of claim 1, wherein the engineered amyloid peptide of step (a) is a β-sheet.

3. The method for preparing the composition for screening coronavirus therapeutic agents of claim 1, wherein step (b) is heating a mixture of hydrogen tetrachloroaurate and ultrapure water, and then adding sodium citrate and heating the mixture.

4. The method for preparing the composition for screening coronavirus therapeutic agents of claim 1, wherein the engineered amyloid nanocomposite of step (c) is prepared by mixing gold nanoparticles and an engineered amyloid peptide in a volume ratio of 1:0.5 to 10.

5. The method for preparing the composition for screening coronavirus therapeutic agents of claim 1, wherein the engineered amyloid nanocomposite of step (c) is red.

6. A composition for screening coronavirus therapeutic agents prepared by the preparation method of claim 1.

7. A method for screening for coronavirus therapeutic agents, comprising treating the composition of claim 6 with a coronavirus therapeutic agent candidate drug.

8. The method for screening for coronavirus therapeutic agents of claim 7, further comprising:
selecting the candidate drug as a coronavirus therapeutic agent if a color changes to blue after the candidate drug treatment.
